(19) United States
(12) Patent Application Publication
(10) Pub. No.: US 2016/0300017 A1
(43) Pub. Date: Oct. 13, 2016

(54) METHOD AND APPARATUS FOR PROVIDING SURGERY-RELATED ANATOMICAL INFORMATION

(71) Applicants: ELECTRONICS AND TELECOMMUNICATIONS RESEARCH INSTITUTE, Daejeon (KR); INDUSTRY-ACADEMIC COOPERATION FOUNDATION, YONSEI UNIVERSITY, Seoul (KR)

(72) Inventors: Jeong Won LEE, Daejeon (KR); Sung Hee PARK, Daejeon (KR); Ji Wook JEONG, Daejeon (KR); Woo Jin HYUNG, Seoul (KR); Soo Yeul LEE, Daejeon (KR); Jong Hyun PARK, Daejeon (KR)

(21) Appl. No.: 15/094,928

(22) Filed: Apr. 8, 2016

(30) Foreign Application Priority Data
Apr. 10, 2015 (KR) 10-2015-0051065

(51) Int. Cl. G06F 19/00; A61B 34/10; G06T 11/20; G06T 15/08; G06T 11/60

(52) U.S. Cl. CPC G06F 19/321; G06T 15/08; G06T 11/60; G06T 11/206; A61B 34/10; G06T 2210/41; A61B 2034/105

(57) ABSTRACT

Disclosed is a method for providing anatomical information, including: loading, by an apparatus for providing anatomical information, image information about a patient; obtaining, by the apparatus for providing anatomical information, information about a blood vessel or an organ related to a specific surgery from the image information about the patient; generating, by the apparatus for providing anatomical information, a three-dimensional (3D) model by using the information about the blood vessel or the organ; and generating, by the apparatus for providing anatomical information, a two-dimensional (2D) model in a form of a diagram from the 3D model.

### CROSS-REFERENCE TO RELATED APPLICATION

This application claims priority to and the benefit of Korean Patent Application No. 10-2015-0051065, filed on Apr. 10, 2015, in the Korean Intellectual Property Office, the entire contents of which are incorporated herein by reference in their entirety.

### BACKGROUND

1. Field

The present disclosure relates to a method and an apparatus for providing surgery-related anatomical information, and more particularly, to a method and an apparatus for providing anatomical information, which are capable of expressing an anatomical singularity of a patient in a two-dimensional diagram in a surgery planning stage or an actual surgery stage, and providing information related to the anatomical singularity.

2. Description of the Related Art

A system for surgery planning has been mainly developed for a skeletal system surgery performed in a plastic surgery, an orthopedic surgery, an oral surgery and the like. Particularly, a surgery planning technology has been developed toward photographing an anatomical image of a patient by using an image device, such as Computerized Tomography (CT) and Magnetic Resonance Imaging (MRI), before a surgery, extracting skeleton information about the patient from the photographed image and re-configuring the extracted skeleton information in a three-dimensional (3D) model, and planning a position of a tool (for example, an implant) to be inserted into a body of the patient by using the 3D model or planning a position and a shape of the skeleton to be resected.

However, the surgery planning technology in the related art has a limit in staying at a step of mainly planning an anatomical position and shape of the skeleton system.

Further, the surgery planning technology in the related art provides skeleton system information by using the anatomical model re-configured in the 3D model, so that it is impossible to provide a surgeon with “simple and intuitive” information (the surgery planning technology in the related art focuses on providing anatomical information about a patient by using a complex 3D model in detail, so that too much information is provided to a surgeon, thereby causing confusion to the surgeon).

Accordingly, development of a new surgery planning technology, which is capable of solving the aforementioned problems, has been demanded.

The present disclosure is invented based on the aforementioned technical background, and is invented in order to provide additional technical elements which meet the aforementioned technical demands and those skilled in the art may not easily invent.

### SUMMARY OF THE INVENTION

The present disclosure has been made in an effort to solve the above-described problems associated with the prior art, and provides a technology for providing anatomical information, which is capable of providing anatomical information related to a blood vessel or an organ of a patient related to a specific surgery (or a particular stage of a specific surgery), and “intuitively” providing a user with only anatomical information related to the specific surgery, not all of the anatomical information about the patient.

In the meantime, technical objects to be achieved by the present disclosure are not limited to the aforementioned objects, and may include various technical objects within the scope apparent to those skilled in the art from the contents to be described below.

An exemplary embodiment of the present disclosure provides a method for providing anatomical information, including: loading, by an apparatus for providing anatomical information, image information about a patient; obtaining, by the apparatus for providing anatomical information, information about a blood vessel or an organ related to a specific surgery from the image information about the patient; generating, by the apparatus for providing anatomical information, a three-dimensional (3D) model by using the information about the blood vessel or the organ; and generating, by the apparatus for providing anatomical information, a two-dimensional (2D) model in a form of a diagram from the 3D model.

The 2D model may display depth information about the organ or the blood vessel based on color information or shade information.

The method may further include displaying, by the apparatus for providing anatomical information, the generated 2D model, and displaying variation type information together when the variation type information related to the generated 2D model exists.

The 2D model may include name information about a selectively displayed blood vessel or organ.

The generating of the 2D model may include generating the 2D model in consideration of view point information, direction information, route information, position information, or operation information about the surgery equipment related to the specific surgery.

The generating of the 3D model may include generating the 3D model by additionally considering beginning point or end point setting information about the blood vessel.

The surgery equipment may include endoscope equipment.

The method for providing anatomical information may be implemented in a form of a program and then stored in an electronic device readable recording medium or distributed through a wired/wireless communication network.

Another exemplary embodiment of the present invention provides an apparatus for providing anatomical information, including: an image information managing unit configured to manage image information about a patent; a 3D model generating unit configured to obtain information about a blood vessel or an organ related to a specific surgery from the image information about the patient, and generate a 3D model by using the obtained information about the blood vessel or the organ; and a 2D model generating unit configured to generate a 2D model in a form of a diagram from the 3D model.

The 2D model may display depth information about the organ or the blood vessel based on color information or shade information.

The apparatus may further include a variation type information managing unit configured to identify whether a variation type related to the generated 2D model exists, and manage information related to the variation type.

The 2D model may include name information about a selectively displayed blood vessel or organ.

The 2D model generating unit may generate the 2D model in consideration of view point information, direction information, route information, position information, or operation information about the surgery equipment related to the specific surgery.

The 3D model generating unit may generate the 3D model by additionally considering beginning point or end point setting information about the blood vessel.

The surgery equipment may include endoscope equipment.

The present invention may “intuitively” provide anatomical information about a patient related to a blood vessel or an organ. Particularly, the present invention may display anatomical information about a patient by using “a 2D model in a form of a diagram”, and selectively display only a main blood vessel or organ related to a specific surgery on a generated 2D model and provide name information about the main blood vessel or organ, thereby provide the anatomical information related to the surgery in an intuitive form.

Further, the present invention may provide a variation type information about a patient. Particularly, when variation type information related to a generated 2D model exists, the present invention may display the corresponding information, and may display the variation type information, to which the generated 2D model belongs, and other types of variation type information contrary to the variation type information, to which the generated 2D model belongs.

Further, the present invention may display “3D information required during for a surgery” on a generated 2D model in an intuitive form. Particularly, the present invention may display “relative depth information of blood vessels or organs” required during a surgery by using color difference, shade difference, and the like, and overcome a limit of the 2D model through the depth information.

Further, the present invention may generate and provide an accuracy guaranteed 2D model. Particularly, the present invention may generate a 2D model by using “a 3D model generated based on image information about a patient”, thereby generating and providing an accuracy guaranteed 2D model.

Further, the present invention may generate and provide an intuitive 2D model specialized to a selected “specific surgery (or a particular stage of a specific surgery). Particularly, the present invention may generate a 2D model in consideration of information related to a selected specific surgery, generate a 2D model in consideration of “beginning point setting information” related to a selected specific surgery, and generate a 2D model in consideration of surgery equipment information (view point information, direction information, route information, position information, and operation information about surgery equipment) related to a selected specific surgery, thereby generating and providing an intuitive 2D model specialized to the selected specific surgery.

Further, the present invention may implement an anatomical information providing technology, which may be utilized even in an actual surgery progressing stage, as well as a surgery planning stage. Particularly, the present invention may collect position information about surgery equipment used in a specific surgery in real time and display the collected information on a generated 2D model, thereby providing intuitive anatomical information even in an actual surgery stage.

In the meantime, the effects of the present invention are not limited to the aforementioned effects, and may include various effects within the scope apparent to those skilled in the art from the contents to be described below.

### BRIEF DESCRIPTION OF THE DRAWINGS

Example embodiments will now be described more fully hereinafter with reference to the accompanying drawings; however, they may be embodied in different forms and should not be construed as limited to the embodiments set forth herein. Rather, these embodiments are provided so that this disclosure will be thorough and complete, and will fully convey the scope of the example embodiments to those skilled in the art.

In the drawing figures, dimensions may be exaggerated for clarity of illustration. It will be understood that when an element is referred to as being “between” two elements, it can be the only element between the two elements, or one or more intervening elements may also be present. Like reference numerals refer to like elements throughout.

FIG. 1 is a flowchart illustrating a method for providing anatomical information according to an exemplary embodiment of the present disclosure.

FIG. 2 is a diagram illustrating an example of a 3D model generated based on image information about a patient.

FIG. 3 is a diagram illustrating an example of a 2D model representing anatomical information about a patient.

FIG. 4 is a diagram illustrating an example of anatomical type information providable together with anatomical information about a patient.

FIG. 5 is a diagram illustrating an example of a User Interface (UI) providable by an apparatus for providing anatomical information according to the exemplary embodiment of the present disclosure.

FIG. 6 is a diagram illustrating a configuration of the apparatus for providing anatomical information according to the exemplary embodiment of the present disclosure.

### DETAILED DESCRIPTION OF THE INVENTION

Hereinafter, “an apparatus and a method for providing anatomical information” according to the present disclosure will be described with reference to the accompanying drawings in detail. Described exemplary embodiments are provided so that those skilled in the art may easily understand the technical spirit of the present invention, so that the present invention is not limited by the exemplary embodiments. Further, matters in the accompanying drawings are illustrated for easily describing the exemplary embodiments of the present invention, and may be different from actually implemented forms.

In the meantime, each element expressed below is an example for implementing the present invention. Accordingly, in another implementation of the present invention, another element may be used without departing from the spirit and the scope of the present invention. Further, each element may be wholly implemented only with a configuration of hardware or software, but may also be implemented by a combination of various hardware and software. Further, two or more elements may be implemented together by one hardware or software.

Further, an expression “including elements” is an open expression, and simply indicates that corresponding elements exist, and shall not be understood that additional elements are excluded.

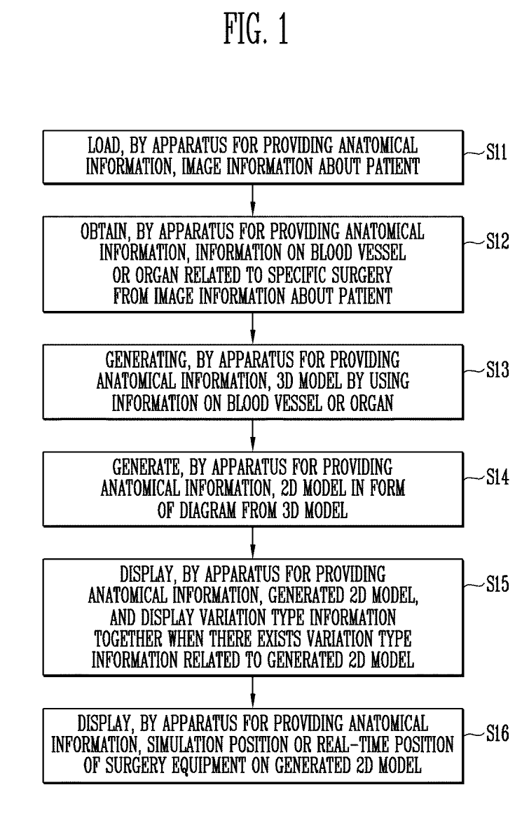

Hereinafter, a method for providing anatomical information according to an exemplary embodiment of the present disclosure will be described with reference to FIGS. 1 to 5.

Referring to FIG. 1, the method for providing anatomical information according to the exemplary embodiment of the present disclosure may include loading, by an apparatus for providing anatomical information, image information about a patient (operation S11), obtaining, by the apparatus for providing anatomical information, information on a blood vessel or an organ related to a specific surgery from the image information about the patient (operation S12), generating, by the apparatus for providing anatomical information, a 3D model by using the information on the blood vessel or the organ (operation S13), generating, by the apparatus for providing anatomical information, a 2D model in a form of a diagram from the 3D model (operation S14), displaying, by the apparatus for providing anatomical information, the generated 2D model, and displaying a variation type information together when there exists the variation type information related to the generated 2D model (operation S15), and displaying, by the apparatus for providing anatomical information, a simulation position or a real-time position of surgery equipment on the generated 2D model (operation S16).

Here, the apparatus for providing anatomical information may be implemented by various kinds of electronic devices, which are capable of performing data processing and displaying visual information.

Further, the apparatus for providing anatomical information may be implemented by a single electronic device including hardware and software, or may be implemented in a form of a system, in which two or more electronic devices are interworked with each other.

Further, the apparatus for providing anatomical information may also be implemented in a form including two or more electronic devices (for example, a “server device” performing main data processing—a “display device” receiving and displaying the processed data) connected with each other through a wired or wireless network, and may be implemented by various electronic devices or system structures, in addition to the aforementioned forms.

Operation S11 is an operation of loading image information about a patent by the apparatus for providing anatomical information.

Here, the apparatus for providing anatomical information may select a specific patient from a plurality of patients or recognize a specific patient based on input information, and search for or receive image information related to the selected or recognized specific patient and then load the image information.

Further, the apparatus for providing anatomical information may load various types of image information representing actual anatomical information about the specific patient. For example, the apparatus for providing anatomical information may load various types of image information containing MRI image information, CT image information, X-Ray image information, and ultrasonic image information, and may also simultaneously load and analyze a plurality of elements of image information in order to improve accuracy of a 3D model or a 2D model to be generated later.

Operation S12 is an operation of obtaining information about a blood vessel or an organ related to the specific surgery from the image information about the patient by the apparatus for providing anatomical information.

Here, the apparatus for providing anatomical information may select a specific surgery (or a particular stage of the specific surgery). Particularly, the apparatus for providing anatomical information may 1) select a “specific surgery” (for which the anatomical information is to be generated) among the plurality of surgeries, or 2) select a “specific surgery” among the plurality of surgeries and then additionally select “a specific particular stage” among the plurality of particular stages included in the specific surgery based on predetermined information or input information.

Further, the apparatus for providing anatomical information may obtain the information about the blood vessel or the organ related to the selected “specific surgery (or particular stage of the specific surgery)”. Particularly, the apparatus for providing anatomical information may extract and obtain the information about the blood vessel or the organ related to the selected “specific surgery” from the image information loaded in operation S11. In this case, the apparatus for providing anatomical information may extract the information about the blood vessel or the organ related to the specific surgery (the particular stage of the specific surgery) in consideration of basic information (for example, surgery region data) about the specific surgery (the particular stage of the specific surgery).

Operation S13 is an operation of generating a 3D model by using the information about the blood vessel or the organ by the apparatus for providing anatomical information.

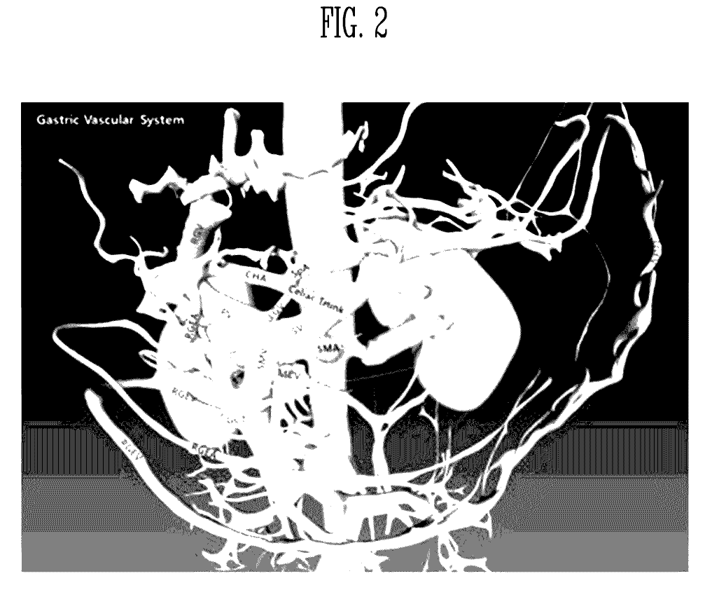

Here, the apparatus for providing anatomical information may render a 3D model based on the information about the blood vessel or the organ related to the “specific surgery” obtained in operation S12. For example, the apparatus for providing anatomical information may generate a 3D model related to the specific surgery as illustrated in FIG. 2. Further, the apparatus for providing anatomical information may also generate a more detailed 3D model for the “specific particular stage” when extracting the information about the blood vessel or the organ “in the unit of the particular stage included in the surgery” in operation S12.

In the meantime, the apparatus for providing anatomical information may also generate the information about the blood vessel included in the 3D model in consideration of a kind (for example, the artery and the vein) or direction information of blood vessel. Further, the apparatus for providing anatomical information may also generate the information about the blood vessel in consideration of the input information or “beginning point setting information” or “end point setting information” set through a data calculation, and make the blood vessels, which need to be essentially displayed in relation to the specific surgery, be included in the 3D model through the generated information about the blood vessel. In the meantime, the number of beginning points or end points may be set to be two or more.

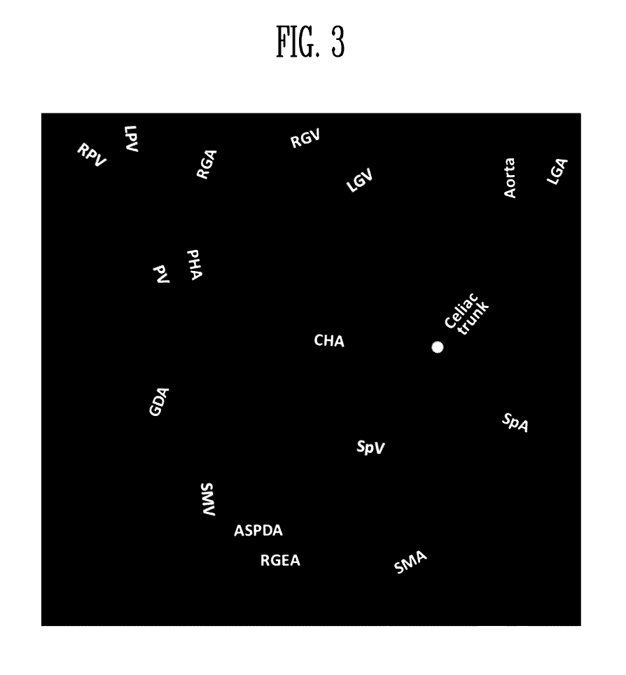

Operation S14 is an operation of generating a “2D model in a form of a diagram” from the 3D model by the apparatus for providing anatomical information. Particularly, operation S14 is an operation of generating a 2D model in the form of a diagram illustrated in FIG. 3 based on the 3D model illustrated in FIG. 2.

Here, the apparatus for providing anatomical information may collect information for generating the 2D model by various methods by using the 3D model generated in operation S13, and generate a “2D model in a form of a diagram” by using the collected information. For example, the apparatus for providing anatomical information may generate the 2D model in the form of the diagram by various methods, such as a “method of projecting the 3D model in a specific direction and then converting the projected 3D model in the form of the diagram” and a “method of analyzing image data of the 3D model and then re-rendering the 2D model in the form of the diagram”. In the meantime, in this case, the information about the blood vessel or the organ included in the 2D model may be further simplified for improving intuitiveness, and information about some blood vessels or organs included in the 3D model may be additionally hided or removed. Further, the model may also additionally display name information about the selectively displayed blood vessel or organ, and thus it is possible to more intuitively provide the important information about the blood vessel or the organ related to the specific surgery through the displayed name information about the selectively displayed blood vessel or organ.

Further, the apparatus for providing anatomical information may display depth information or the blood vessel or the organs included in the 2D model through additional image processing, and overcome a limit of the intuitive 2D model through the displayed depth information. For example, the apparatus for providing anatomical information may display depth information by using a “color difference” as illustrated in FIG. 3, and in addition, may display depth information by using various image processing methods including a “shade difference”.

In the meantime, the apparatus for providing anatomical information may also generate the 2D model from the 3D model in consideration of information about “surgery equipment” used in the specific surgery (or the particular stage included in the specific surgery). For example, the apparatus for providing anatomical information may determine a direction for projecting the 3D model, or extract the information about the blood vessel or the organ, which is included in the 3D model, but needs to be hidden or removed, in consideration of view point information, direction information, route information, position information, operation information, and the like about the surgery equipment, and generate the 2D model based on the operations. In the meantime, herein, the surgery equipment may be surgery equipment injected into the blood vessel or the organ. For example, the surgery equipment may be endoscope equipment, a micro robot, and the like. Accordingly, the 2D model may be implemented in a form of selectively displaying only “the blood vessel or the organ, into which the surgery equipment is directly injected” and “peripheral blood vessels or organs which may be influenced during an injection process or an operation process of the surgery equipment”, thereby helping a safe operation or an effective surgery operation of the surgery equipment.

Operation S15 is an operation of displaying, by the apparatus for providing anatomical information, the generated 2D model in the form of the diagram, and displaying the variation type information together when the variation the information related to the 2D model exists.

Here, the apparatus for providing anatomical information may display the 2D model related to the specific surgery or display the 2D model related to the particular stage included in the specific surgery. Further, the apparatus for providing anatomical information may also display a dynamic 2D model according to a change in particular stages included in the specific surgery.

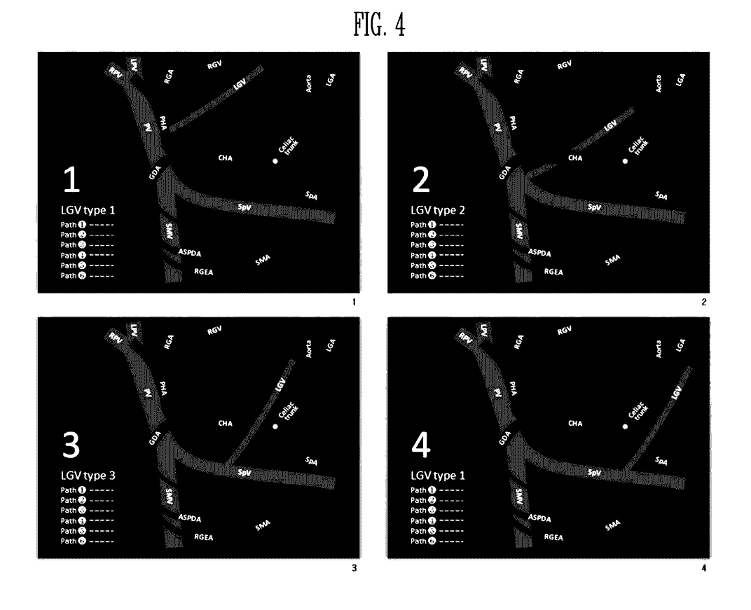

In the meantime, the apparatus for providing anatomical information may determine whether the variation type information related to the generated 2D model exists, and display the corresponding information when the variation type information related to the generated 2D model exists. For example, the apparatus for providing anatomical information may display the plurality of variation types related to the generated 2D model as illustrated in FIG. 4, and display information about a type, to which the generated 2D model belongs, among the plurality of variation types.

Operation S16 is an operation of displaying a simulation position or a real-time position of the surgery equipment related to the specific surgery on the 2D model by the apparatus for providing anatomical information.

Here, the apparatus for providing anatomical information may display a simulation position of the surgery equipment related to the specific surgery on the 2D model, or a simulation position of the surgery equipment related to the particular stage included in the specific surgery on the 2D model. Further, the apparatus for providing anatomical information may also display the simulation position of the surgery equipment in a dynamic form according to a change in the particular stages included in the specific surgery.

Further, the apparatus for providing anatomical information may also receive real-time position information about the surgery equipment related to the specific surgery, and display the received real-time position information on the generated 2D model. Accordingly, the apparatus for providing anatomical information may provide intuitive surgery information even during an actual surgery process, as well as the surgery planning.

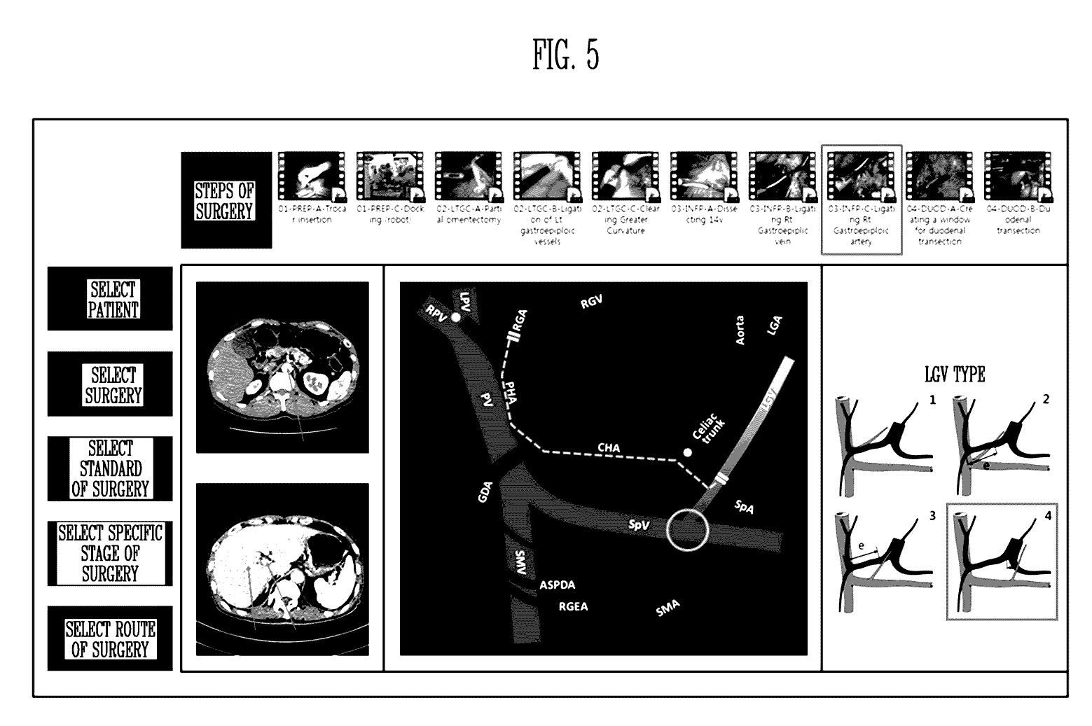

In the meantime, the method for providing anatomical information may be provided through various User Interfaces (UIs). For example, the method for providing anatomical information may provide the method for providing anatomical information through various UI environments including an UI environment illustrated in FIG. 5.

Further, the method for providing anatomical information may be implemented in a form of a program and then stored in a recoding medium readable by the electronic device or may be distributed in a form downloadable by various types of electronic devices through a wired/wireless communication network.

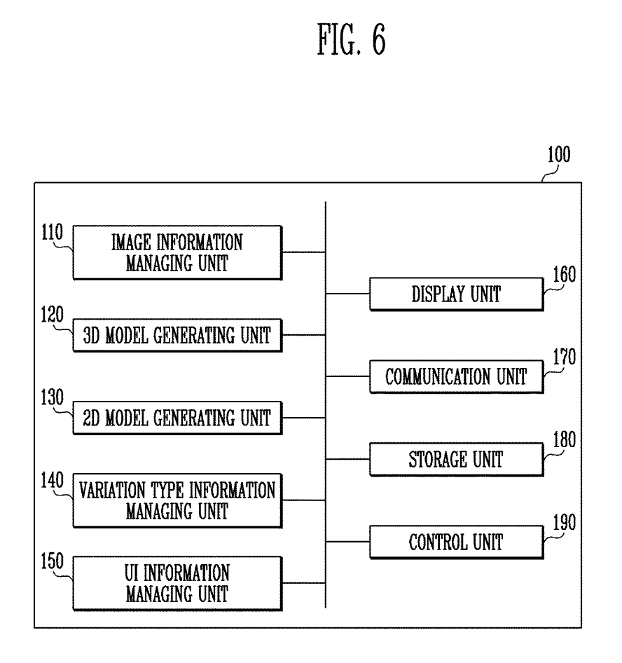

Hereinafter, an apparatus 100 for providing anatomical information according to an exemplary embodiment of the present disclosure will be described with reference to FIG. 6.

For reference, the apparatus 100 for providing anatomical information according to an exemplary embodiment of the present disclosure to be described below may be included in a different category from that of the apparatus for providing anatomical information according to the exemplary embodiment of the present disclosure described above, but include the substantially same (or corresponding) technical characteristic as that of the apparatus for providing anatomical information according to the exemplary embodiment of the present disclosure described above. Accordingly, in order to prevent the overlapping description, although not described in detail, the various technical characteristics described in relation to the method for providing anatomical information may be applied to the apparatus 100 for providing anatomical information to be described below.

Further, the elements to be described below may be wholly implemented only with a configuration of hardware or software as mentioned at the beginning, but may also be implemented by a combination of various hardware and software. Further, two or more elements may be implemented together by one hardware or software.

Referring to FIG. 6, the apparatus 100 for providing anatomical information according to the exemplary embodiment of the present disclosure may include an image information managing unit 110, a 3D model generating unit 120, a 2D model generating unit 130, a variation type information managing unit 140, an UI information managing unit 150, a display unit 160, a communication unit 170, a storage unit 180, and a control unit 190.

The image information managing unit 110 is a configuration of managing image information about a patient. For example, the image information managing unit 110 may select a specific patient among the plurality of patients based on input information and the like, and may load image information related to the selected specific patient.

The image information managing unit 110 may perform the various aforementioned operations related to the management of the image information about the patient.

The 3D model generating unit 120 is a configuration of obtaining information about a blood vessel or an organ related to a specific surgery (or a specific particular stage included in a specific surgery) from the image information about the patient, and generating a 3D model by using the obtained information about the blood vessel or the organ.

The 3D model generating unit 120 may generate the 3D model by additionally considering beginning point or end point setting information about the blood vessel, and in addition to the operations, may perform the aforementioned various operations related to the generation of the 3D model.

The 2D model generating unit 130 is a configuration of generating a 2D model in a form of a diagram from the 3D model.

The 2D model generating unit 130 may generate the 2D model in consideration of view point information, direction information, route information, position information, operation information, and the like of the surgery equipment related to the specific surgery, generate the 2D model displaying depth information about the organ or the blood vessel based on color information or shade information, generate the 2D model including name information about the selectively displayed blood vessel or organ, or generate the 2D model displaying real-time position information about the surgery equipment, and in addition to the operations, may perform the aforementioned various operations related to the generation and the display of the 2D model.

The variation type information managing unit 140 is a configuration of identifying whether a variation type related to the generated 2D model exists, and managing information related to the variation type.

The variation type information managing unit 140 may perform the aforementioned various operations related to the variation type.

The UI information managing unit 150 is a configuration of managing a UI environment of a user related to the method for providing anatomical information. The UI information managing unit 150 may manage various types of UI data including FIG. 5.

The display unit 160 is a configuration of displaying various information related to the method for providing anatomical information. The display unit 160 may be implemented through various kinds of display devices.

The communication unit 170 is a configuration of transceiving various data related to the method for providing anatomical information through a wired/wireless communication network. The communication unit 170 may transceive various kinds of data with various electronic devices, and particularly, may receive real-time information (real-time position information and the like) about the surgery equipment from the surgery equipment related to the specific surgery. In the meantime, the communication unit 170 may be implemented in a form including various kinds of wired communication modules or wireless communication modules.

The storage unit 180 is a configuration of storing various information related to the method for providing anatomical information. For example, the storage unit 180 is a configuration storing various information including the image information about the patient, the information about the organ or the blood vessel related to the specific surgery extracted from the image information about the patient, the beginning point or end point setting information, user input information, the information about the generated 3D model, the information related to the surgery equipment related to the specific surgery, the information about the generated 2D model in the form of the diagram, and the variation type information. The storage unit 180 may be implemented in a form including various kinds of memory devices.

The control unit 190 is a configuration of controlling various operations of the apparatus 100 for providing anatomical information including the operations of the image information managing unit 110, the 3D model generating unit 120, the 2D model generating unit 130, the variation type information managing unit 140, the UI information managing unit 150, the display unit 160, the communication unit 170, and the storage unit 180.

The control unit 190 may include at least one calculation device, and herein, the calculation device may include a common Central Processing Unit (CPU), a programmable device element (Complex Programmable Logic Device (CPLD), Field Programmable Gate Array (FPGA)) implemented to be suitable to a specific purpose, a semiconductor calculation device on demand (Application Specific Integrated Circuit (ASIC)), or a micro controller chip.

As described above, even though the disclosure made by the inventor has been described in detail with the exemplary embodiment, it is apparent that the present disclosure is not limited to the exemplary embodiment, but various modifications and changes may be made without departing from the main principle of the present disclosure.

What is claimed is:

1. A method for providing anatomical information, comprising:
loading, by an apparatus for providing anatomical information, image information about a patient;
obtaining, by the apparatus for providing anatomical information, information about a blood vessel or an organ related to a specific surgery from the image information about the patient;
generating, by the apparatus for providing anatomical information, a three-dimensional (3D) model by using the information about the blood vessel or the organ; and
generating, by the apparatus for providing anatomical information, a two-dimensional (2D) model in a form of a diagram from the 3D model.

2. The method of claim 1, wherein the 2D model displays depth information about the organ or the blood vessel based on color information or shade information.

3. The method of claim 1, further comprising:
displaying, by the apparatus for providing anatomical information, the generated 2D model, and displaying variation type information together when the variation type information related to the generated 2D model exists.

4. The method of claim 1, wherein the 2D model includes name information about a selectively displayed blood vessel or organ.

5. The method of claim 1, wherein the generating of the 2D model includes generating the 2D model in consideration of view point information, direction information, route information, position information, or operation information about the surgery equipment related to the specific surgery.

6. The method of claim 1, wherein the generating of the 3D model includes generating the 3D model by additionally considering beginning point or end point setting information about the blood vessel.

7. The method of claim 1, wherein the surgery equipment includes endoscope equipment.

8. An electronic device readable recording medium, in which a program for implementing a method for providing anatomical information is recorded, the method comprising:
loading, by an apparatus for providing anatomical information, image information about a patient;
obtaining, by the apparatus for providing anatomical information, information about a blood vessel or an organ related to a specific surgery from the image information about the patient;
generating, by the apparatus for providing anatomical information, a three-dimensional (3D) model by using the information about the blood vessel or the organ; and
generating, by the apparatus for providing anatomical information, a 2D model in a form of a diagram from the 3D model.

9. An apparatus for providing anatomical information, comprising:
an image information managing unit configured to manage image information about a patent;
a 3D model generating unit configured to obtain information about a blood vessel or an organ related to a specific surgery from the image information about the patient, and generate a 3D model by using the obtained information about the blood vessel or the organ; and
a 2D model generating unit configured to generate a 2D model in a form of a diagram from the 3D model.

10. The apparatus of claim 9, wherein the 2D model displays depth information about the organ or the blood vessel based on color information or shade information.

11. The apparatus of claim 9, further comprising:
a variation type information managing unit configured to identify whether a variation type related to the generated 2D model exists, and manage information related to the variation type.

12. The apparatus of claim 9, wherein the 2D model includes name information about a selectively displayed blood vessel or organ.

13. The apparatus of claim 9, wherein the 2D model generating unit generates the 2D model in consideration of view point information, direction information, route information, position information, or operation information about the surgery equipment related to the specific surgery.

14. The apparatus of claim 9, wherein the 3D model generating unit generates the 3D model by additionally considering beginning point or end point setting information about the blood vessel.

15. The apparatus of claim 9, wherein the surgery equipment includes endoscope equipment.
